(19) United States
(12) Patent Application Publication
(10) Pub. No.: US 2020/0107788 A1
(43) Pub. Date: Apr. 9, 2020

(54) CARDIOVASCULAR RISK SCREENING CATEGORIZATION USING BLOOD PRESSURE MONITOR VARIABLE INACCURACY

(71) Applicant: PACIFIC DELTA LLC, Butte, MT (US)

(72) Inventor: Stephen Christopher HIMLEY, Butte, MT (US)

(21) Appl. No.: 16/153,000

(22) Filed: Oct. 5, 2018

(51) Int. Cl. A61B 5/00; A61B 5/022

(52) U.S. Cl. CPC A61B 5/7275; A61B 5/022; A61B 5/7221; A61B 2560/0214; A61B 5/0004; A61B 5/743; A61B 2560/0223; A61B 5/7425

(57) ABSTRACT

Systems and methods for using blood pressure monitoring instrument inaccuracy to accomplish categorization based on the instrument's measurements and inaccuracy in conjunction with a categorization assessment algorithm are disclosed. Incorporation of inaccuracy enables high confidence screening for cardiovascular risk based on biomarker and other data within risk criteria algorithms, and also incorporating blood pressure monitoring instrument measurement with inaccuracy. Screening results can be displayed as a confidence figure above or below a risk threshold, or via assignment into one of various risk categories around a threshold, with the categories determined in part by apparatus inaccuracy.

### TECHNICAL FIELD

Disclosed embodiments relate to health risk screening; specifically, apparatuses and methods for achieving high confidence in cardiovascular risk screening outcomes based in part on blood pressure instrument measurement confidence are disclosed.

### BACKGROUND OF THE INVENTION

The global prevalence of hypertension has been estimated at 1.39 billion adults in 2010 (Mills K. et al. Global Disparities of Hypertension Prevalence and Control: A Systematic Analysis of Population-based Studies from 90 Countries. Circulation. 2016 Aug. 9; 134(6): 441-450. doi:10.1161/CIRCULATIONAHA.115.018912). It is responsible for at least 45% of deaths due to heart disease and 51% of deaths due to stroke (WHO. A global brief on Hypertension—Silent killer, global public health crisis. WHO/DCO/WHD/2013.2).

### BRIEF DESCRIPTION OF THE DRAWINGS

FIG. 1 is a diagram showing a blood pressure measuring apparatus configuration with the measurement unit integrated with a display/control unit and attached to a blood pressure interface unit, according to some embodiments.

FIG. 2 is a diagram showing a blood pressure measuring apparatus in a mobile system configuration with the display/control unit separate from the measurement unit that is attached to a blood pressure interface unit (shown as a brachial arm cuff and bulb in this example), according to various embodiments.

FIG. 3 is a diagram showing one possible architecture for a blood pressure measuring apparatus that maintains status of measurement unit inaccuracy, according to various embodiments.

FIG. 4 is a diagram showing an example screening categorization with three categories, screened out, screened in, and urgent, based on predetermined thresholds and on an inaccuracy value of one standard deviation, according to various embodiments.

FIG. 5 is a diagram showing an example screening categorization with four categories, screened out, indefinite, screened in, and urgent, based on inaccuracy value of one standard deviation, according to various embodiments.

FIG. 6 is a flowchart of one possible method for screening categorization when the apparatus displays blood pressure and screening category that may be carried out in part or in whole by a blood pressure measuring apparatus such as those disclosed in FIGS. 1 and 2, according to various embodiments.

FIG. 7A shows an example categorization screening outcome of screened out, for a client whose actionable systolic blood pressure threshold is 160 mmHg, the apparatus measurement is 155 mmHg, and the current measurement unit inaccuracy is a standard deviation of 5 mmHg, according to various embodiments.

FIG. 7B shows an example categorization screening outcome of indefinite, for a client whose actionable systolic blood pressure threshold is 160 mmHg, the apparatus measurement is 156 mmHg, and the current measurement unit inaccuracy is a standard deviation of 5 mmHg, according to various embodiments.

FIG. 7C shows another example categorization screening outcome of indefinite, for a client whose actionable systolic blood pressure threshold is 160 mmHg, the apparatus measurement is 160 mmHg, and the current measurement unit inaccuracy is a standard deviation of 5 mmHg, according to various embodiments.

FIG. 7D shows yet another example categorization screening outcome of indefinite, for a client whose actionable systolic blood pressure threshold is 160 mmHg, the apparatus measurement is 164 mmHg, and the current measurement unit inaccuracy is a standard deviation of 5 mmHg, according to various embodiments.

FIG. 7E shows an example categorization screening outcome of screened in, for a client whose actionable systolic blood pressure threshold is 160 mmHg, the apparatus measurement is 165 mmHg, and the current measurement unit inaccuracy is a standard deviation of 5 mmHg, according to various embodiments.

FIG. 8A shows an example graphical way to display inaccuracy, depicting a solid box indicating good or high accuracy, according to various embodiments.

FIG. 8B shows an example graphical way to display inaccuracy, depicting a partially filled box showing moderate accuracy, according to various embodiments.

FIG. 8C shows an example graphical way to display inaccuracy, depicting a nearly empty box showing poor or low accuracy, according to various embodiments.

### DETAILED DESCRIPTION OF THE INVENTION

The International Society for Hypertension (ISH) reports that only half of all people with hypertension are aware of their condition. This lack of awareness is especially severe in countries without high-income. Assessment for and diagnosis of hypertension occurs virtually entirely within clinical facilities, especially in countries without high income, i.e. lower resource settings (LRS). Measurement of blood pressure within clinical facilities is performed using blood pressure (BP) monitor devices of various types including more modern digital BP monitors, which can be re-calibrated at the recommended intervals with appropriate clinical engineering support.

Digital BP monitors may have inaccuracies in their measurements from day one of use, where a typical new unused device has a specification of the readings having inaccuracy up to +/−3 mmHg (millimeters mercury). The inaccuracy of a new unused device can vary with pressure range, typically within 2% error at higher BP readings, e.g. a systolic BP reading of 200 mmHg would have inaccuracy up to +/−4 mmHg. After the devices are used for some time, the inaccuracies may grow. In a University of Ottawa study, 210 nephrology patients brought their home digital BP monitors to clinic where their readings were compared to clinical BP measurements using standard mercury sphygmomanometers (Ruzicka M. et al. How Accurate Are Home Blood Pressure Devices in Use? A Cross-Sectional Study. PLOS One, DOI:10.1371/journal.pone.0155677 Jun. 1, 2016). Of these 210 digital BP monitors, 63 (30%) had systolic BP measurements that were 5 mmHg or more different from the standard, and 16 (8%) had systolic BP measurements that were 10 mmHg or more different from the standard.

Fitting these data to a normal (Gaussian) distribution of errors, these 210 digital BP monitors had a collective standard deviation of 5.2 mmHg. Thus, these users could have only 44% confidence that their actual blood pressure was within the manufacturers' specified +/−3 mmHg of their digital BP monitor reading. This growth of inaccuracy of digital BP monitors over time is ideally accounted for when using BP measurements for clinical decision making.

A further complication of digital BP monitor readings can occur when they are used in non-ideal environments. BP monitors are qualified for use in air-conditioned room temperature and moderate humidity conditions. However, in various settings, such as found in LRS, BP measurements may be made in environments that are colder or hotter, and across a wider range of humidity conditions, than specified for the BP monitor being used. Knowing the inaccuracy of a given BP monitor in non-ideal settings is important when making diagnostic and treatment decisions.

The typical recommended re-calibration period for digital BP monitors is every 1-2 years. For many settings, this translates into loss of confidence in device accuracy after only 1 to 2 years, due to the prohibitive costs or logistical burdens of re-calibration. Particularly in LRS, it may be cost-prohibitive to re-calibrate a digital BP monitor, since this typically requires sending each unit back to the factory in another country for re-calibration. Re-purchase of new equipment every 1 to 2 years is also cost-prohibitive.

In high-income countries, patients continue to use their devices without re-calibration. These patients could keep a qualitative check of their home BP monitor readings by comparing them with the clinical readings during their periodic clinic checkups. When home BP monitor readings become far different from the clinic readings, this could signal replacement of their home BP monitors. However, the different settings of home and clinic produce naturally different BP readings, which makes this qualitative judgment difficult for patients to assess reliably.

After 1 to 2 years, the lack of confidence in accurate BP readings limits usage of home digital BP monitors to relative monitoring, which can be backed up with clinical support via frequent clinical checkups using calibrated BP monitors in clinical facilities. In LRS, where conditions are non-ideal, re-calibration is virtually non-existent, and medical equipment are used as long as apparently functional, home digital BP monitors used as primary clinical diagnostic instruments have large errors in their readings after only 1 or 2 years, with progressively larger errors over time. However, the clinical users of these devices do not know their BP monitors have become inaccurate, because the displays continue to show BP numbers which they assume are still valid.

A digital BP monitor that lasts several years with known current inaccuracy values would be beneficial for determining confidence that can be use in risk screening. The inventor has submitted U.S. patent application Ser. No. 16/137,198, filed on 20 Sep. 2018, the contents of which is hereby incorporated by this reference in its entirety, which is directed to similar such methods and apparatuses. Such a BP monitor can incorporate information about its inaccuracy over the lifetime of the device, so the user knows how confident they can be in the displayed numbers. This inaccuracy may also account for the operating environment and the physiologic/acquisition conditions of the reading, i.e. larger error at higher blood pressure readings and low heart rate and high deflation rate for pneumatic type BP technologies. By including inaccuracy information over time to provide confidence in the readings, the useful life of a digital BP monitor in these settings could be extended from just 1 or 2 years to several years, with significantly decreased annualized equipment cost. This is particularly attractive in LRS where awareness of hypertension is very low and a very large number of people may require screening and basic treatment. Such a long-life blood pressure monitor would enable community screening and identification of these people with hypertension and high cardiovascular risk.

A variety of different algorithms can be used for screening to determine if someone is at risk of developing a disease, such as heart disease or stroke. These algorithms permit assessment of a person's risk using multiple clinical and socio-demographic data for each person (client being assessed). The World Health Organization (WHO) and ISH have developed cardiovascular (CV) risk prediction charts, which are used in the WHO Package of Essential Non-Communicable Diseases Interventions (PEN). These charts identify persons at high CV risk. For example, for a particular WHO sub-region and gender of client (not yet a patient) who is a smoker and age 50 without diabetes, the chart could show a threshold of 160 mmHg for establishing greater than 40% chance of major cardiovascular event in the next 10 years. Such a client should be treated for hypertension and potentially other issues, especially when they have a blood pressure in excess of 160 mmHg. With this single value threshold, a BP monitor reading of 160 mmHg would include the patient for treatment, while a reading of 159 mmHg would exclude her or him.

However, given the typical device error of 5 or 10 mmHg for a digital BP monitor as described above, use of a BP reading from such a digital BP monitor at face value could result in either under- or over-treatment. Using the standard deviation of 5.2 mmHg noted above for real world BP monitors, a reading of 162 mmHg would have a 35% probability that the client's actual blood pressure was less than the 160 mmHg threshold and only a 65% probability of correctly screening the client in. Viewed another way, the client has a 35% chance of being prescribed a course of treatment, which may include various pharmaceuticals, that is unnecessary, resulting in unnecessary drug expenses and possible side effects that otherwise could have been avoided. Potentially more dangerous is the client who has a reading of 159 mmHg with a digital BP monitor that has the aforementioned standard deviation of inaccuracy. Such a client has a non-negligible chance of having an actual blood pressure that exceeds the 160 mmHg screen-in threshold, but will not otherwise receive treatment due to the relatively inaccurate digital BP monitor reading. Thus, the client may actually be high risk, but may not receive recommended, possibly life-saving treatment. Considering the foregoing, the inaccuracy of a BP monitor reading should be incorporated into screening algorithms for diagnosis and treatment decisions. This is facilitated by having a knowledge of a BP monitor's current level of accuracy, and a method for incorporating inaccuracy into screening algorithms.

A screening device that reliably identifies those at high risk and reliably excludes those at low risk is desirable. Accurate diagnosis of hypertension benefits from repeat BP measurements, preferably over multiple days, and is best performed outside of clinic facilities to avoid masking and white-coat effects. However, in LRS, the costs of accessing clinic services even for one visit are prohibitive except for urgent or emergent conditions, potentially making multiple visits for screening purposes cost-prohibitive. Thus, a BP monitor where lay persons can perform repeat BP measurements with a reasonable degree of confidence, at the community level, in client homes or nearby their homes, is useful to accurately detect cases of hypertension that should be treated.

An object of the present invention is to provide a method and an apparatus for using blood pressure measuring instrument inaccuracy to accomplish cardiovascular risk categorization based on the apparatus' measurements and inaccuracy in conjunction with a categorization assessment algorithm. The disclosed example apparatus and method enable high confidence screening for cardiovascular risk based on biomarker and other data within risk criteria algorithms, while also incorporating blood pressure measurement with inaccuracy.

One exemplary embodiment uses an inaccuracy value for the current blood pressure monitor measurement to provide a confidence figure that the reading is above or below a threshold.

Another possible embodiment uses the inaccuracy values of a blood pressure monitor in determining risk categorization for public health screening. Any established risk criteria algorithm could be used, for example an algorithm that estimates risk based on client data such as biomarker data, behavior data, and demographic data. Screening results are displayed via assignment into one of various risk categories around a threshold, with the categories determined in part by blood pressure monitor measurement inaccuracy.

According to one or more embodiments of the claimed invention, by integrating risk screening algorithms and blood pressure measuring instrument inaccuracy over time, effective screening with high confidence can be accomplished under non-ideal conditions affecting the instrument measurement. This enables effective public health screening using blood pressure instrument measurements to identify persons with high cardiovascular risk.

In the following description, various aspects of the illustrative implementations will be described using terms commonly employed by those skilled in the art to convey the substance of their work to others skilled in the art. However, it will be apparent to those skilled in the art that embodiments of the present disclosure may be practiced with only some of the described aspects. For purposes of explanation, specific numbers, materials, and configurations are set forth in order to provide a thorough understanding of the illustrative implementations. However, it will be apparent to one skilled in the art that embodiments of the present disclosure may be practiced without the specific details. In other instances, well-known features are omitted or simplified in order not to obscure the illustrative implementations.

In the following detailed description, reference is made to the accompanying drawings that form a part hereof, wherein like numerals designate like parts throughout, and in which is shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

For the purposes of the present disclosure, the phrase “A or B” means (A), (B), or (A and B). For the purposes of the present disclosure, the phrase “A, B, or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

It is the express intention that the claims are not to be construed in a means plus function form unless a claim being construed explicitly recites the words “means”, “means to”, or “means for.” Absent the word “means”, language such as “unit” or “module” is not to be construed as means plus function.

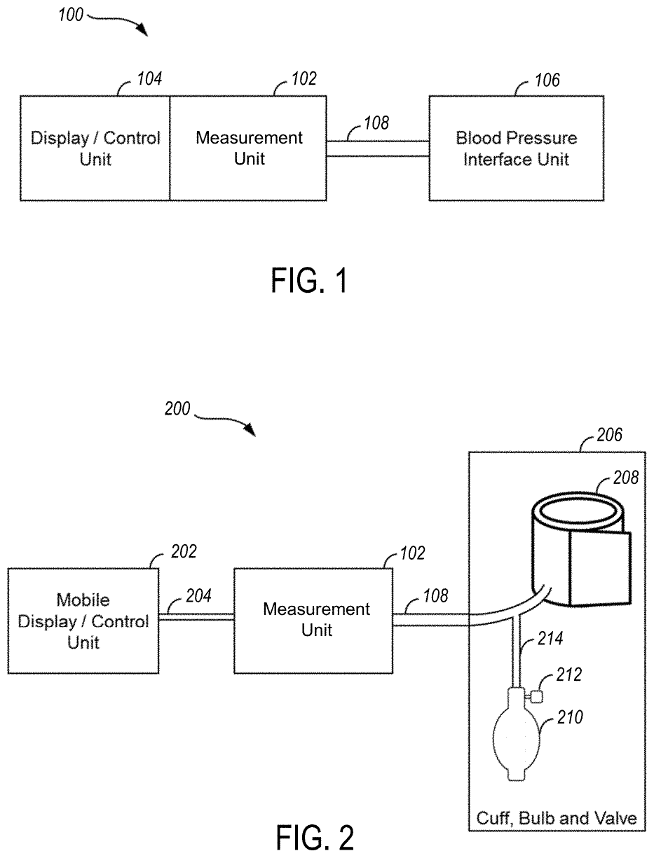

FIG. 1 shows an apparatus 100 useable with disclosed risk screening algorithms according to a first possible embodiment in a configuration, where a measurement unit 102 includes an integrated display/control unit 104. The measurement unit 102 is in turn in communication via communications link 108 with a blood pressure interface unit 106. The measurement unit 102 will be further described below. The blood pressure interface unit 106 could be any non-invasive blood pressure instrument technology, such as automatic inflating wrist cuff, brachial (arm) blood pressure measurement using auscultation or oscillometric technology, volume clamping technology, pulse transit time technology, or cuff-less finger pressure with photoplethysmography oscillometry technology, to name a few examples. Any technology now known or later developed for measuring blood pressure suitable for use with a measurement unit 102 as described herein may be employed.

Integrated display/control unit 104 can serve to provide various controls and monitoring for measurement unit 102. For example, display/control unit 104 may allow for triggering the start of a blood pressure measurement, where blood pressure interface unit 106 provides for an automated measurement cycle, e.g. an automatic inflating wrist cuff. In another example, display/control unit 104 may be used to signal the measurement unit 102 to begin recording or monitoring data from blood pressure interface unit 106, such as where blood pressure interface unit 106 is manually operated, e.g. a manual inflating/deflating cuff and bulb. Display/control unit 104 may further provide an interface for various functions and/or status of apparatus 100, such as performing diagnostic routines, displaying device usage history, environmental conditions, etc. Other functionality may include reconfiguring measurement unit 102 to accommodate a different style or type of blood pressure interface unit 106, where measurement unit 102 is configured to accept multiple types of blood pressure measurement equipment, and providing a user with instructional guidance for properly measuring blood pressure with the equipped blood pressure interface unit 106. Display/control unit 104 may provide information about device inaccuracy as well as screening categories, as will be described in greater detail herein.

Display/control unit 104 may include an interface comprised of one or more displays to convey information to a user, as well as one or more ways by which the user may interact with display/control unit 104. Such interface may include a touch screen to provide both information to the user, as well as accepting input in the form of touches and/or gestures. Other examples may have a non-touch enabled screen or other type of display, e.g. LED, LCD, lamps, and similar indicators, along with one or more buttons, switches, sliders, etc., for accepting input. The particular configuration of display/control unit 104 for a given embodiment may depend upon the type of blood pressure interface unit 106 with which apparatus 100 is equipped. As described above, in apparatus 100 the display/control unit 104 is integrated and part of measurement unit 102, and so may share various components of measurement unit 102.

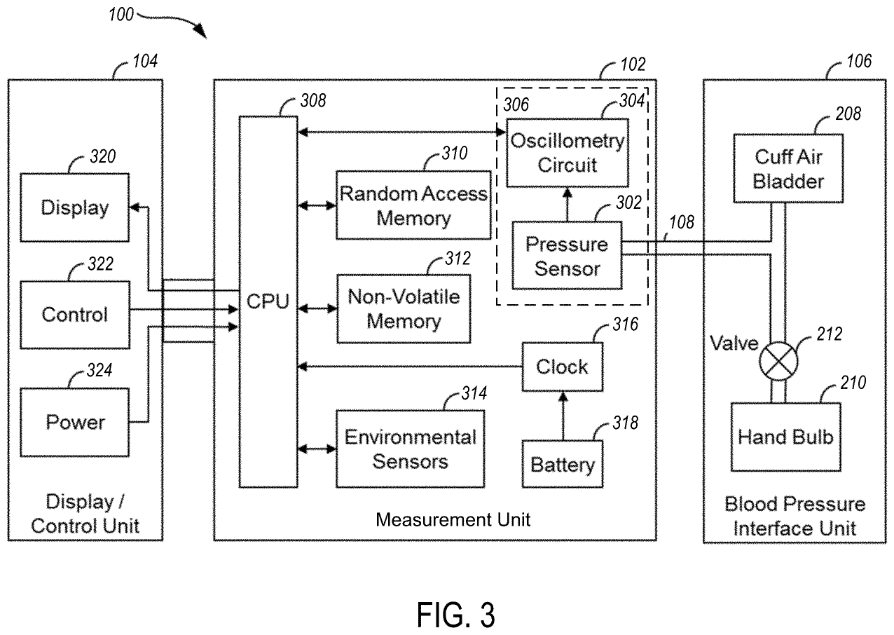

FIG. 2 shows an apparatus 200 according to a second possible embodiment equipped with a mobile display/control unit 202 that is external to and discrete from measurement unit 102, and connected to the measurement unit 102 by a communications link 204. Communications link 204 may be either wired or wireless, and may utilize any communications technology suitable to support communications between mobile display/control unit 202. Such technologies may include wireless technologies such as WiFi, Bluetooth, NFC, Zigbee, or other similar protocols. In one example, display/control unit 202 may be implemented as a dedicated interface unit, specific to measurement unit 102, and similar in implementation to display/control unit 104, save for being discrete from measurement unit 102. In another example, display/control unit 202 could be implemented in software, executable on a generic device such as a computer, mobile phone, laptop, tablet, wearable device such as a smart watch, or any other similarly suitable computing device now known or later developed.

Display/control unit 202 in apparatus 200 provides identical functionality to display/control unit 104 of apparatus 100. As used herein, “display/control unit 104” is intended to refer to embodiments either with the integrated display/control unit 104, or with the mobile display/control unit 202. As with the embodiment depicted in FIG. 1, display/control unit 202 can supply system power, display, and control of the data acquisition cycle performed by the measurement unit 102. Supply of power, control and display communication can be via a cable 204 or by wireless communication. In other embodiments, measurement unit 102 may have an independent power supply and/or control unit.

Blood pressure interface unit 206 may be implemented similar to blood pressure interface unit 106 of FIG. 1. The blood pressure measurement interface unit 206 in the example embodiment depicted in FIG. 2 is a brachial arm or wrist cuff and hand bulb. As shown, blood pressure interface unit 206 includes an inflating cuff 208, configured to go around a patient's arm or wrist. An inflation bulb 210 is pneumatically connected to inflating cuff 208 via air hose 214. A valve 212 is interposed on air hose 214 between inflation bulb 210 and inflating cuff 208 to control the inflation of inflating cuff 208. As will be understood by a person experienced in operating a blood pressure measurement cuff, when valve 212 is closed, air is forced from inflation bulb 210 into inflating cuff 208 when inflation bulb 210 is squeezed, thereby inflating the cuff. When valve 212 is opened, the air under pressure within inflating cuff 208 is released, causing inflating cuff 208 to deflate. The speed at which inflating cuff deflates may depend upon the extent to which valve 212 is opened. As depicted, blood pressure interface unit 206 may be operated manually. In such an embodiment, apparatus 200 may be configured to receive blood pressure measurements upon signaling by an operator, who would then proceed to inflate and deflate the inflating cuff 208 according to known procedures for measuring blood pressure.

In other embodiments, inflation bulb 210 and valve 212 may be automated to various extents, to allow automatic inflation and deflation of inflating cuff 208, such as where apparatus 200 is configured to automatically measure the blood pressure of a patient. For example, inflation bulb 210 may instead be implemented as a motorized air pump or compressor, with valve 212 being automatically actuated. In such embodiments, measurement unit 102 may control the automation of blood pressure interface unit 206 to effect automatic measurement of blood pressure. A display/control unit 104 may allow the operator to trigger the automatic cycle for measuring blood pressure.

Blood pressure interface unit 106 and 206 each may connect to measurement unit 102 via a communications link 108, which, in various embodiments, may be a physical hard link (e.g. cable, fiber optics, tube, etc.), or via a wireless connection using any suitable communications technology. Such implementations may be used where measurement unit 102 receives the measurement numbers from blood pressure interface unit 106, which itself handles the actual measuring and quantifying of the physical signals (e.g. pressure changes, optical changes, etc.) from the blood pressure measurement. In other embodiments, such as where measurement unit 102 includes a module or functionality for performing the actual measuring and quantifying of blood pressure, communications link 108 may conduct physical signals, e.g. air pressure, hydraulic pressure, light, etc., as appropriate to the specific implementation of blood pressure interface unit 106/206. For example, in embodiments where measurement unit 102 includes a sensing unit necessary to acquire blood pressure measurements from the attached blood pressure interface unit (discussed below), communications link 108 may be implemented to carry physical signals to the sensing unit. In the embodiment depicted in FIG. 2, where blood pressure interface unit 206 includes an inflating cuff, communications link 108 is implemented as a pneumatic hose, with the sensing unit of measurement unit 102 configured to measure air pressure changes, the air pressure changes reflecting blood pressure. In still other embodiments, communications link 108 may carry both physical signals as well as communications, such as where measurement unit 102 includes a sensing unit, and blood pressure interface unit 106 is automated to allow for automatic capture of blood pressure measurements.

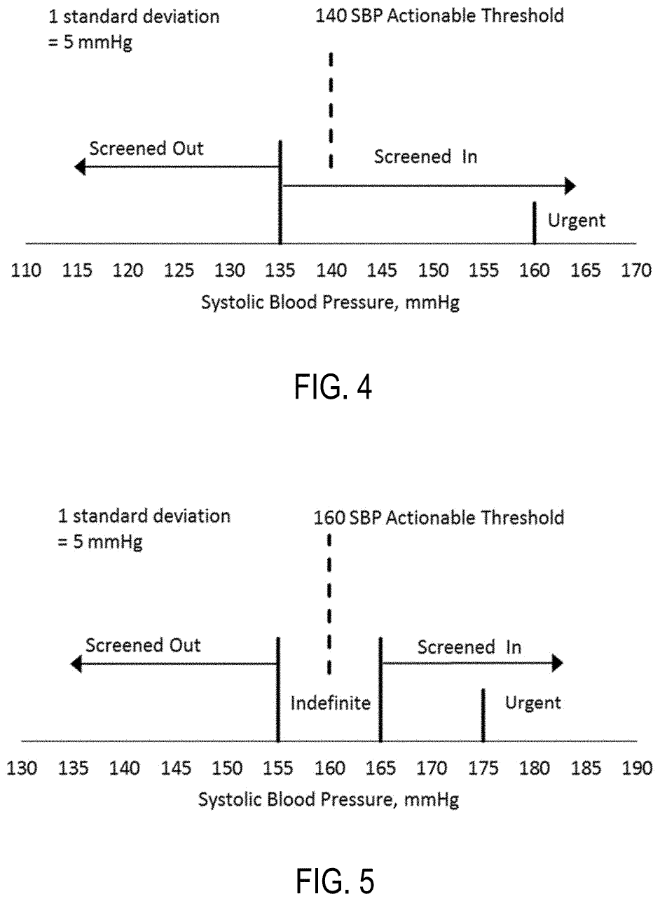

FIG. 3 is a block diagram showing the architecture of a blood pressure monitoring apparatus according to various embodiments that monitors measurement unit 102 inaccuracy status and displays results accordingly. In the example depicted in FIG. 3, the blood pressure interface unit 106 includes an inflating cuff 208 and a hand-actuated inflation bulb 210 with valve 212, described above with respect to FIG. 2. Inflating cuff 208 contains an air bladder that is connected to a pressure sensor 302 within the measurement unit 102, which measures air pressure from communications link 108 continuously while an oscillometry circuit 304 determines at what points the air pressure reflects systolic blood pressure, mean blood pressure, and diastolic blood pressure, using simple or complex analysis in any state-of-the-art fashion to determine these points. The combination of pressure sensor 302 and oscillometry circuit 304 comprises a sensing unit 306, as described above with respect to FIGS. 1 and 2. Sensing unit 306 may, in other embodiments, be equipped with different modules that are appropriate to a particular implementation of blood pressure interface unit 106. In embodiments where blood pressure interface unit 106 includes integrated sensors and so delivers measurement numbers to measurement unit 102, sensing unit 306 may instead simply be a receiver for measurement data coming from blood pressure interface unit 106, via communications link 108.

In embodiments, measurement unit 102 may contain a central processing unit (CPU) 308 that receives power from and communicates with the display/control unit 104. CPU 308 may be in communication with various types of storage, including volatile random access memory (RAM) 310 available for processing as well as non-volatile memory (NVM) 312 that does not lose information when power is disconnected. CPU 308 controls initiation of the sensing unit 306 and its data acquisition. CPU 308 further monitors and collects information from environmental sensors 314, which, in various embodiments, can include thermal, humidity, barometric pressure, and acceleration sensors. The choice of RAM 310 and NVM 312 may depend upon the selection of CPU 308, as well as any operating system or application-specific software that measuring unit 102 may use. NVM 312 may comprise, at least in part, firmware or other storage that contains any instructions or other software for operation of measuring unit 102. RAM 310 may be suitable for allowing CPU 308 to efficiently execute any firmware or software stored in NVM 312.

CPU 308 may be any suitable processor capable of carrying out and/or controlling or arbitrating the functions of sensing unit 306, and/or executing any firmware or software that may be stored in NVM 312. Examples of possible implementations of CPU 308 include general purpose processors, such as those available from ARM or Intel, an application-specific integrated circuit (ASIC), a field-programmable gate array (FPGA), discrete components, a combination of any of the foregoing, or another suitable implementation for performing the functions of measuring unit 102. It should further be understood that various components of measurement unit 102 may be integrated on to a single component, e.g. a System on a Chip (SoC) implementation as may be found in many ARM implementations and embedded controllers, such as those available from ATMel. SoC implementations may integrate one or more of RAM 310, NVM 312, CPU 308, clock 316, and environmental sensors 314 onto a single or a few components.

Environmental sensors 314 may include other sensors appropriate to a given implementation of measurement unit 102. Such sensors may include temperature, humidity, vibration/shock (such as with an accelerometer or gyroscope), particulates/atmospheric contamination, atmospheric pressure (such as with a barometer) and/or sensors for measuring any other environmental conditions that may affect the accuracy of apparatus 100. Such sensors may be implemented using any components now known or later developed.

A clock 316 and battery 318, preferably of a long-life type, can provide information on the age of the apparatus 100 since first operation. Clock 316, as used here, may be a real-time clock, as opposed to an oscillator for driving CPU 308. A real-time clock may allow tracking of total system age, as well as total system operational time, which may be used to help determine how age and operational time factors into device inaccuracy.

Various possible blocks in display/control unit 104 are also depicted in FIG. 3. As described above, display/control unit 104 may include a display 320 for providing operational status and/or instructions to a user. A control 322 may allow a user to manipulate various aspects of apparatus 100, such as triggering measurement of blood pressure, running diagnostics, viewing use history, environmental conditions, setting one or more screening thresholds, etc. As described above, control 322 may be integrated with display 320 in various embodiments, such as where display 320 is a touch screen (e.g. where display/control unit 104 is implemented as an app on a smartphone, with a touchscreen). In other embodiments, control 322 may be discrete from display 320. Display/control unit 104 may further include a power source 324, which may, in various embodiments, power not only the display/control unit 104 but also measuring unit 102 and/or blood pressure interface unit 106. In some embodiments, display/control unit 104 may be a smartphone, tablet, computer, or other similar device, running appropriate software and connected to measurement unit 102, such as by a wireless connection like Bluetooth. Other embodiments may attach the device to measurement unit via a USB or other hard-wired interface, particularly where display/control unit 104 is to provide power to measurement unit 102 and/or blood pressure interface unit 106. Still further, display/control unit 104 may allow the user to enter client data and thresholds (discussed below), and may inform the user via the display when apparatus 100 is ready for the user to inflate and then deflate the cuff (in the example depicted in FIGS. 2 and 3) in order to acquire a blood pressure reading.

In various embodiments, information stored in the NVM 312 may include inaccuracy characterization data determined for the specific model and/or lot number of measurement unit 102. Such inaccuracy characterization data may be calculated by the manufacturer of measurement unit 102 based on quality control and/or related testing of sample measurement units 102. The inaccuracy characterization data can provide a general curve of inaccuracy and/or degradation of measurement unit 102 that is inherent into a given design and production run of measurement unit 102. The inaccuracy characterization data may reflect degradation of measurement unit 102, current state of measurement unit 102 inaccuracy, and the categorization assessment algorithm discussed below. The inaccuracy characterization data may comprise the inaccuracy function described below, an equation or look-up table that is a function of time (age of the measurement unit 102), history of blood pressure measurement cycles, and the current environmental operating condition: temperature, humidity, barometric pressure, and vibration. Such data may be provided by clock 316 as well as environmental sensors 314.

With time and wear, measurement unit 102 inaccuracy may become progressively larger. In some embodiments, the current state of measurement unit 102 inaccuracy is updated using the inaccuracy function following each blood pressure measurement cycle, and the current inaccuracy may be stored in NVM 312. In other embodiments, measurement unit 102 inaccuracy may be computed on the fly with each test cycle on the basis of stored information in NVM 312, such as number of measurement/test cycles, environmental conditions experienced with each cycle, age of the measurement unit 102 determined from clock 316, and any other factors relevant or contributing to progressive inaccuracy of measurement unit 102. NVM 312 may further store information of previous calibrations, such as the last time the apparatus 100 was calibrated, the amount of correction required, the tolerance/inaccuracy of apparatus 100 following calibration, and other similar parameters or data points.

As discussed above, the accuracy of apparatus 100 (or 200) may decrease over time for a number of different factors. Measurement unit 102 can be configured to track, determine, and characterize the inaccuracy of a particular measurement at any time in the lifetime of the measurement unit 102. For some embodiments, it is not necessary to characterize each individual measurement unit 102, rather each model of measurement unit 102 for a given application. The inaccuracy of the measurement unit 102 may consist of contributions both dependent and independent of the current operating condition. The total inaccuracy of the measurement unit 102 for any given blood pressure reading at any given point in the life of the unit may be the combined contributions to inaccuracy of factors both dependent and independent of the current operating condition.

Some possible contributions to inaccuracy that are independent of current operating conditions include the aging of the internal components and the cycle history of these internal components. In embodiments, this history consists of the number of blood pressure measurement cycles experienced by the measurement unit 102. Since many sensors are composed at least partially of non-inert materials, these materials are subject to non-reversible physical changes when exposed over time to environmental factors such as temperature, humidity, pressure differences, and vibration. For many embodiments, temperature and humidity may have the greatest effect. These environmental factors can cause sensor response to change over time, thereby causing inaccuracy in readings compared to when measurement unit 102 was last calibrated. The cycle history may be modified by the particular environmental history seen by the measurement unit 102, with, for example, a larger inaccuracy value for units exposed to cycles at more severe temperatures and/or humidity levels than units exposed only at room temperature or to moderate humidity.

Another possible contribution to inaccuracy is, for implementations where blood pressure interface unit 206 is implemented as depicted in FIG. 2 and apparatus 200, a fluid overpressure event where the fluid sensor (e.g. pressure sensor 302/sensing unit 306) is exposed to very high pressure, such as an air pressure sensor used in oscillometry. The exposure to such events can be monitored and its contribution to inaccuracy included. Alternatively, physical means to protect the sensor against overpressure events can be included, for example, by incorporating a relief valve or other means to limit the pressure that the sensor experiences. It should be understood that apparatuses that implement blood pressure interface unit 106 using other technologies, e.g. light sensing, may have inaccuracies impacted by different conditions specific to the particular technology used in the given implementation.

Determination and characterization of the current inaccuracy of measurement unit 102, in one example, may include using the pressure sensor characteristic drift over time, typically in the range of 0.3 mmHg to 1.8 mmHg per year for high quality pressure sensors operating in the range of 0 to 300 mmHg. Other examples may include life-testing of the same make and model of measurement unit 102 under the various environmental conditions that affect accuracy of the unit. Typically, accelerated testing is conducted at extreme environmental conditions. The testing may result in creation of inaccuracy curves, an equation or look-up table that is a function of time (age of the measurement unit 102), and history of blood pressure measurement cycles at the environmental operating conditions of each cycle: temperature, humidity, barometric pressure, and vibration. Each model of measurement unit 102 requires testing, since different constellations of components produce different inaccuracy functions. More refined characterization could also be performed, such as characterization of measurement unit 102s for each lot of primary pressure sensor.

Any statistic and symmetric or asymmetric error distribution could be used for the inaccuracy value, but for illustration purposes and in various embodiments, the inaccuracy value used herein is a standard deviation (SD) of a normal (Gaussian) distribution, with equal positive and negative errors of a reading. The SD and normal distribution allow determination of the probability of error at given confidence levels by calculating the area under the distribution curve. Various statistical confidence levels could also be used, for example the +/− error could be one SD or 2 SDs or 3 SDs corresponding to 68% or 95% or 99.7% confidence respectively that the measurement is within the error bounds. In other embodiments, the inaccuracy may be expressed as a tolerance range.

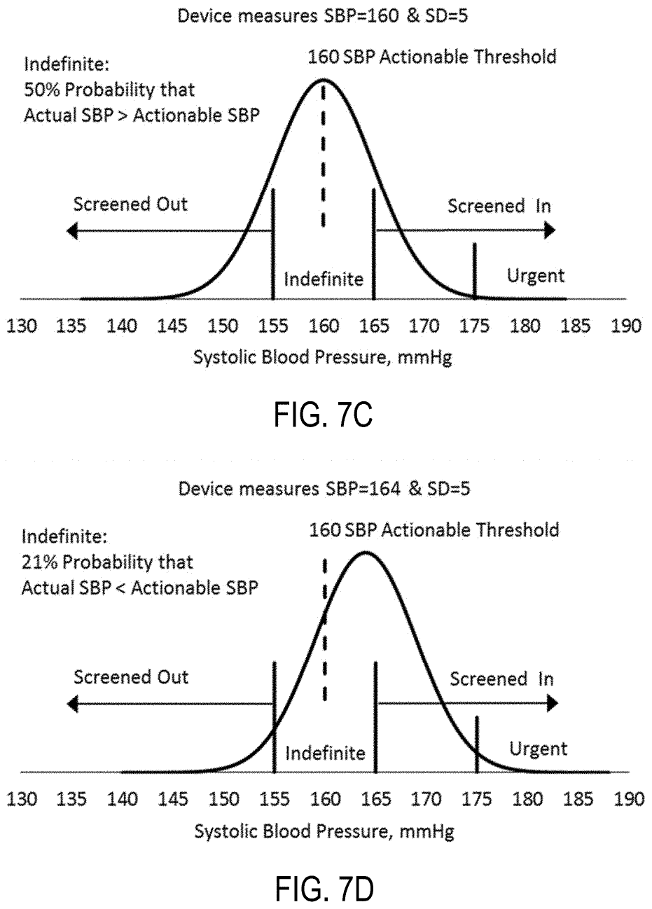

The measurement unit inaccuracy value is used in combination with a categorization algorithm, described below with respect to FIG. 6, to accomplish screening of clients for inclusion into or exclusion out of further clinical evaluation and potential treatment for blood pressure-related CV risk factors. Screening of a client to assess cardiovascular (CV) risk, as discussed above, can be based on an actionable systolic blood pressure value, when other factors are known or assumed, such as gender, age, smoking status, global sub-region of residence, and others. One or more of these other factors may be client factors, which may be obtained directly from a client, such as via an intake questionnaire. In some embodiments, such a questionnaire may be supplied directly to the client via display/control unit 104 or 202, or via a device such as a smartphone, tablet, or computer (which itself may act as at least part of display/control unit 104, as discussed above).

One such categorization assessment algorithm is represented by the WHO/ISH 10 year cardiovascular risk categorization tables. If a health system adopts a 10 year cardiovascular risk level, for example >30% or >40% risk as inclusion for treatment, potential clients can be identified as those whose systolic blood pressure and other factors place them above that risk level. Given particular client factors, the systolic blood pressure level associated with the high CV risk level for treatment can be used as that client's actionable systolic blood pressure. Through blood pressure screening, if a client is found to have a systolic blood pressure higher than the actionable systolic blood pressure based on her or his other factors, that client is identified for further clinical assessment to verify high CV risk and initiate therapeutic interventions.

In the examples to follow, only one risk level is assumed with a corresponding single actionable blood pressure. However, the apparatus could present results for multiple actionable blood pressures, for example the actionable blood pressure levels corresponding to >20% CV risk, >30% CV risk, and >40% CV risk. Each actionable level may be associated with a specific predetermined blood pressure threshold, as will be understood herein.

Screening can be performed at the community or village level with a display/control unit 104 or 202 that has external communication ability, for example, with a smart phone. When a client has been identified as having a systolic blood pressure higher than the actionable systolic blood pressure, the apparatus 100 or 200 could prompt the user to send the current measured data to a receiving referral center, along with client identification information that the user would enter. Such referral center, which may be part of a local or regional health system, could engage the client for further evaluation and a possible course of treatment and/or monitoring.

In some embodiments, the actionable threshold is determined from the categorization assessment algorithm, after the client factors are entered into the apparatus prior to initiating a blood pressure measurement. For example, a non-smoking woman age 50 years residing in WHO sub-region SEAR-D might have a high (40% or greater) 10-year risk of a major cardiovascular event if her systolic blood pressure is 160 mmHg or higher, given other assumed factors. In this example, the apparatus 100 measures systolic blood pressure, determines the current measurement unit inaccuracy value, and then displays a confidence value for the client being above the actionable threshold. For example if the actionable threshold for the client is 160 mmHg, and the measurement unit measures the client's systolic blood pressure as an estimated 164 mmHg with a current inaccuracy value being a standard deviation of 2 mmHg, then the apparatus 100 would display that there is 95% confidence that the client is above the threshold for 40% cardiovascular risk, since the estimated systolic blood pressure is two SDs above the threshold. Similarly, if the same apparatus 100 were to estimate the same client's systolic blood pressure as 156 mmHg, the apparatus 100 would display that there is 95% confidence that the client is below the threshold; alternatively the apparatus 100 could display that there is only a 5% confidence that the client is above the threshold, or present both confidence values above and below the threshold. In all cases, the client could make a decision whether to engage further treatment on the basis of the displayed confidence.

In another embodiment, the apparatus 100 displays the category of screening for the client, based on an actionable predetermined threshold and predetermined confidence levels around that threshold. Categorization could be as simple as two categories, such as in/out or above/below the threshold, using a fixed confidence value above the predetermined threshold as the determinant of inclusion. For example, client inclusion might be determined whenever the apparatus 100 estimates systolic blood pressure to be above the actionable threshold with more than 80% confidence given its current inaccuracy value. The confidence can be calculated using a standard probability equation for the normal distribution and can also be determined using Z-score transformation tables. In such embodiments, the client may not be notified of the confidence level, but only whether the client is screened in or screened out. In other embodiments, the user may also be informed of the confidence level, with the screen in or screen out determined by the combination of actionable threshold and confidence level. In still other embodiments, the user may select a threshold for the confidence level based on his or her personal comfort in any potential risk of mistreatment.

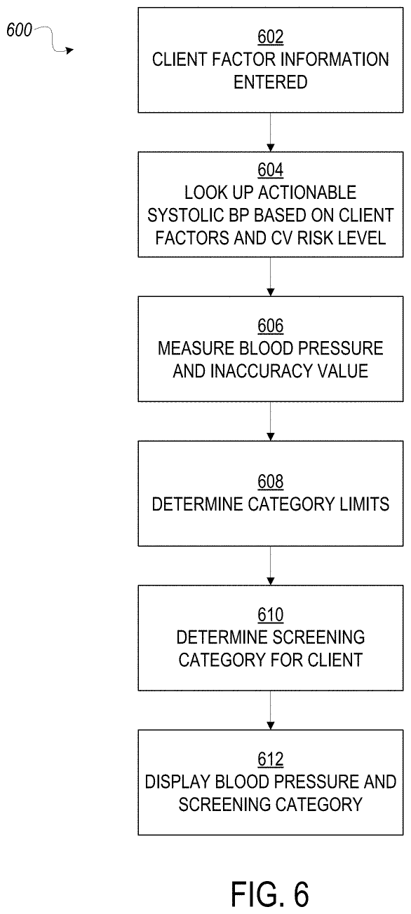

Multiple categories may be implemented in some embodiments to add urgency and nuance to the screening process. FIG. 4 shows an example of screening into three categories based on inaccuracy value of one standard deviation, with a Screen-Out category being greater than 1 SD below the actionable threshold, Screen-In being 1 SD below the actionable threshold and higher, and Urgent category when estimated BP is an Urgent Delta above the actionable threshold. The Urgent category would be used to identify clients who should have urgent action taken due to very high blood pressure. For example, if the actionable threshold is 140 mmHg and an Urgent Delta is set at 20 mmHg, the client whose systolic blood pressure was estimated over 160 mmHg would be Screened-In and also identified as Urgent with very high blood pressure. The selection of one SD for delineation of categories below or above the actionable threshold is shown for example purpose only.

In embodiments based on the depiction in FIG. 4, a first predetermined threshold of 135 mmHg is established based upon the 140 mmHg actionable threshold, and a second predetermined threshold at 160 mmHg is established for urgent action. The first threshold of 135 mmHg is determined as a combination of the actionable threshold combined with the standard deviation of 5 mmHg. Put another way, the predetermined threshold of 135 mmHg is determined based on the actionable threshold combined with a predetermined acceptable percentage of deviation, based upon the standard deviation and/or an acceptable percentage of confidence. It will be appreciated that a client may change the first predetermined threshold, either directly or by changing the acceptable percentage of confidence. In some implementations, the actionable threshold of 140 mmHg may be coincident with the predetermined threshold. Likewise, the second predetermined threshold of urgent may also be determined based at least partially on the standard deviation or some percentage of confidence. In embodiments, the second predetermined threshold may be associated with a different acceptable percentage of confidence than the first predetermined threshold.

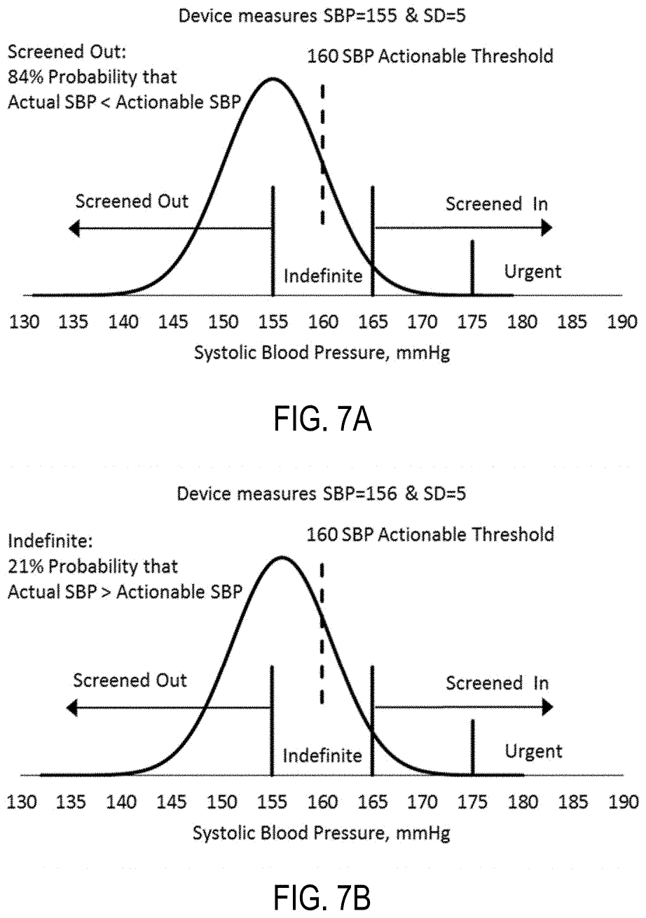

FIG. 5 shows an example of screening into four categories based on an inaccuracy value of one standard deviation, with screen-out category being greater than 1 SD below the actionable threshold, Indefinite being from 1 SD below to 1 SD above the actionable threshold, screen-in being 1 SD above the actionable threshold and higher, urgent category when estimated BP is an urgent delta above the actionable threshold. In this example, the urgent delta is 15 mmHg and the actionable threshold for this client is a systolic blood pressure of 160 mmHg. The indefinite category allows for local health system determination of action. More aggressive health systems may wish to refer all indefinite clients for referral and further clinical evaluation. Less aggressive health systems may wish to defer further clinical action on indefinite clients, preferring instead to re-screen after some time period. The indefinite category enables greater confidence that those in the screen-in and screen-out categories are correctly identified. The selection of one SD for delineation of categories below or above the actionable threshold is shown for example purpose only.

As with the example of FIG. 4, in FIG. 5 the first predetermined threshold for screened out of 155 mmHg and second predetermined threshold for screened in of 165 mmHg are based on the combination of the actionable threshold of 160 mmHg in combination with the standard deviation or a first acceptable percentage of confidence. Likewise, the urgent setting, as a third predetermined threshold, may be based on a second acceptable percentage of confidence, which may be the same as the first acceptable percentage of confidence.

FIG. 6 shows a flowchart for a method 600, to be carried out in whole or in part with an apparatus 100 (or 200), where the client is categorized relative to the actionable blood pressure threshold. Method 600 may be carried out at least in part with software that is executed on a display/control unit 104, such as on a smartphone or tablet when used as a display/control unit 104. Method 600 may further be carried out on a separate, standalone computer. In some embodiments, portions of method 600 may be carried out on apparatus 100, with other portions carried out on an external system, such as a computer system that may be in communication with apparatus 100.

In initial operation 602, client factor information is entered, as described above. The client factor information may be input directly into apparatus 100, via a mobile display/control unit 202, or into a separate computer system that may be configured to receive BP readings from apparatus 100. Prior to use with any clients, each apparatus 100 may initially be set up with geographic region of use and the CV risk level upon which action should be taken, with set-up typically performed by the local or regional health system. The apparatus 100 user thus only needs to enter client factor information, such as gender, age, smoking status, and diabetes status if known. Further, in operation 602 a client (or their attending medical personnel) may also enter predetermined thresholds or acceptable percentages of confidence for the various screening categories. After the user enters the client information in operation 602, in operation 604 the apparatus 100 or other implementing system looks up the actionable systolic blood pressure corresponding to the client factors.

In operation 606, in embodiments, the apparatus 100 displays a message to the user to inflate the cuff and slowly release pressure, which directions may be provided on display/control unit 104. The apparatus 100 measures the client blood pressure, and the measurement unit 102 determines its inaccuracy value. The measurement unit 102 then determines category limits based on the actionable threshold and the inaccuracy value.

Following measurement of blood pressure, in operation 608 apparatus 100 determines the thresholds for the various screening categories, such as where the predetermined thresholds are expressed in terms of acceptable percentages of confidence. In operation 610, apparatus 100 determines which screening category (e.g. screened in, screened out, indefinite/indeterminate, urgent, etc.) the client falls into and displays the client's nominal blood pressure and the client's screening category, based upon the measured BP in combination with the determined inaccuracy value as described above, in comparison to the predetermined thresholds calculated in operation 608.

Following determination of the screening category, in operation 612 the determined category may be provided to the user/client. In some embodiments, the accuracy range or percentage of confidence may further be displayed to the user/client. A referral action is optional at this point. If the screening category is “Screened-In”, the apparatus 100 can prompt the user to enter further client information and then communicate via text or email or other means to the referring clinical center. Immediate referral helps establish the public health referral loop between the clinical center and the community or village of the client and screening personnel.

It should be understood that, although the various operations of method 600 detailed above are described as being displayed and/or performed by an apparatus 100, any of the steps may be performed on a separate system. For example, entry of factors in operation 602 may be performed on a computer at a clinic during initial client intake, and then uploaded to an apparatus 100. Likewise, the display of information from operation 612 may be performed on a separate computer, such as a laptop, which may be in communication or receive an upload from apparatus 100. Other steps may be performed in whole or in part by various other systems.

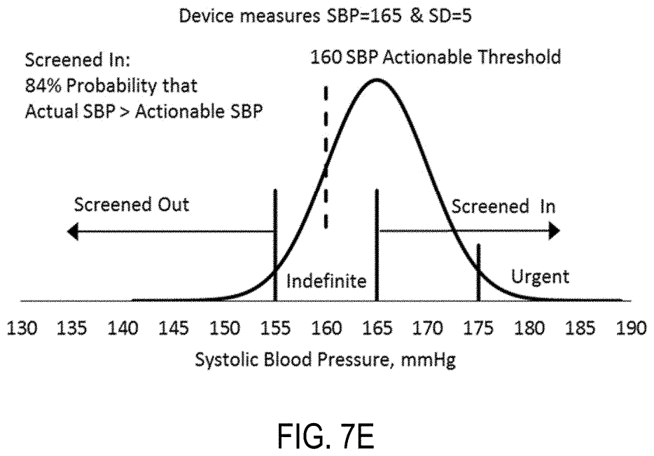

The beneficial effects of combining apparatus 100 inaccuracy with screening categories are shown in the examples of FIGS. 7A-7E, where the actionable threshold for a particular client is a systolic blood pressure of 160 mmHg, and the current measurement unit 102 inaccuracy when measuring blood pressure near this value under the current operating conditions is a standard deviation of 5 mmHg. Superimposed on the screening categories is the example apparatus' 100 measurement reading and its distribution curve of possible actual blood pressure values given that reading and its current standard deviation of 5 mmHg. The area under the curve above or below any particular blood pressure value is the probability that the client's actual blood pressure is above or below that value. For example, in FIG. 7A, the apparatus measures systolic blood pressure as an estimated 155 mmHg, with a probability of 84% that the client's actual blood pressure is less than the actionable blood pressure, corresponding to the proportion of the area under the curve being below 160 mmHg. In this case, the client is in the screened out category. In FIG. 7B, the apparatus measures systolic blood pressure as an estimated 156 mmHg, which is just high enough to screen the client in the indefinite category, with a 21% probability that the client's actual blood pressure is greater than 160 mmHg. It will be understood that the percentage of probability is the same as the aforementioned acceptable percentage of confidence, equating to a predetermined inaccuracy range.

FIG. 7C shows the case where the apparatus 100 estimates systolic blood pressure to be 160 mmHg, equal to the actionable threshold value. The screening outcome is indefinite, with 50% probability that the client's actual blood pressure is above the threshold and 50% probability that the client's actual blood pressure is below the threshold. Note that in the current state of the art, a blood pressure monitor with a similar current inaccuracy of 5 mmHg that measured systolic blood pressure of 160 mmHg would screen the client in, but with only a 50% probability of being correct; there would be a 50% probability that the client should have been screened out since that is the probability the actual blood pressure would be less than 160 mmHg. By at least supplying the inaccuracy range (e.g. 50%), a client can make a more informed choice about whether to seek treatment based upon their comfort with a 50% confidence level.

FIG. 7D shows the high end of being screened as indefinite, when the apparatus 100 estimates systolic blood pressure as 164 mmHg, having a 21% probability that the actual blood pressure is less than 160 mmHg. FIG. 7E shows the case where the apparatus 100 estimates systolic blood pressure as 165 mmHg, resulting in being in the screened in category, with an 84% probability that the client's actual systolic blood pressure is greater than the actionable systolic blood pressure of 160 mmHg. In the case where the apparatus estimates systolic blood pressure above the urgent level (175 mmHg in the prior examples), the screening outcome is screened in and also urgent. It will be observed that, for some embodiments, an urgent category screening may result only when the percentage of confidence for the screened in category is well past 90% range, viz. it is nearly certain that the client is screened in and should seek medical treatment.

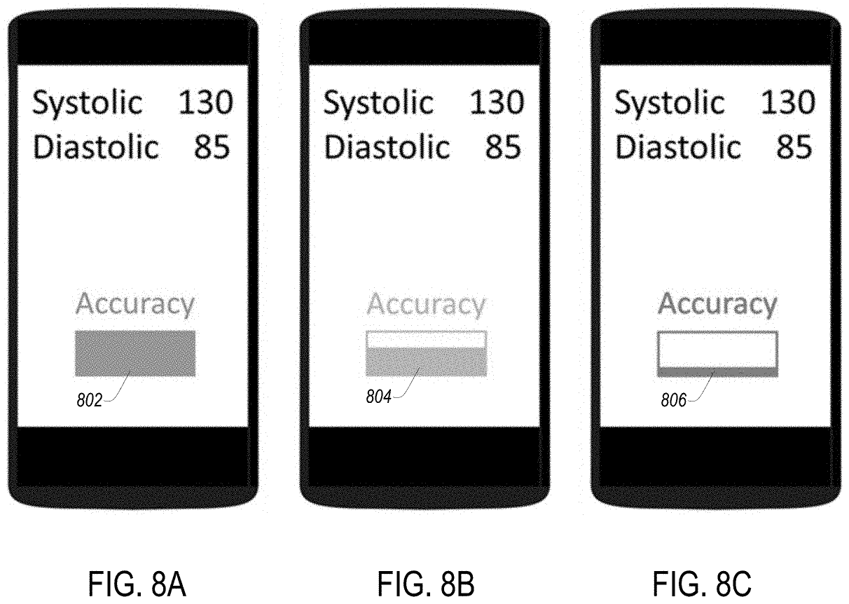

FIGS. 8A-8C show an embodiment that employs a relatively simple means of relaying category or accuracy information to the user, presented using a fuel or battery gauge metaphor reflecting accuracy, with a color or shading potentially reflecting the screened in/screened out/indefinite categories. FIG. 8A shows a display where inaccuracy is low/accuracy is high, indicated with a filled box 802. The box could be colored green, indicating a screened out category. FIG. 8B shows a display where inaccuracy is moderate, indicated with a partially filled box 804. The box could be colored orange or yellow to indicate an indefinite category. FIG. 8C shows a display where inaccuracy is high/accuracy is low, indicated with a nearly empty box 806. The box could be colored red to indicate a screened in category. Alternately, orange or yellow could indicate screened in, with red indicating urgent. In lieu of or in addition to colors, each box could use a different pattern to indicate various screening categories. In still other embodiments, a combination of colors, such as alternating green/yellow, or a fourth color, could be employed to represent indefinite screening.

It will be understood that the combinations of colors and level of fill may be mixed across the three depicted displays, e.g. a box could have full, partial, or empty boxes, representing the degree of inaccuracy, in combination with any of the colors to represent the screening category determined from the measured BP. For example, a partially filled box that is green may indicate a screened out category, but based upon a low accuracy or percentage of confidence. Likewise, a fully filled box that is green may indicate a screened in or urgent category that is determined with a high degree of confidence.

Warning messages could accompany these indicators, such as direction to recalibrate or service or replace the device. The box further could be configured to be continuously variably filled, rather than just a three-step depiction (full, mid, and empty), with the level of fill reflecting the actual inaccuracy range. Other embodiments may forego changing the fill of the box, such as where the inaccuracy range or percentage of confidence is not displayed, and instead present a completely filled box, like box 802, with the color or fill pattern of the box changing to represent the screening category, similar to a stop light. Still other embodiments may use a changing fill or pattern, rather than or in addition to color, to represent the screening category and/or accuracy; such an embodiment may be useful for people who have deficient color vision that prevents them from easily distinguishing between red, orange, and green.

As inaccuracy grows over time with ageing of the apparatus, the size of the indefinite category may grow. For example, if SD becomes 10 mmHg, the size of the indefinite category would become 20 mmHg wide. This may result in fewer clients falling within the screened out or screened in categories, and more clients falling into the indefinite category. To maintain value in the screening method, inaccuracy information can be displayed so the user can observe progressive growth of inaccuracy with apparatus ageing. Various ways can be used to display inaccuracy, as described in U.S. patent application Ser. No. 16/137,198, referenced above.

Categorization could occur via processing within the measurement unit itself or via processing within the associated display/control unit 104. Multiple mobile display/control unit 104s could be attached to the same measurement unit at different times. This would enable different users to measure blood pressure by connecting the measurement unit and blood pressure interface unit to their own mobile display/control unit 104s, for example their own smart phones.

Note that the embodiments disclosed above are understood to be in all ways exemplary and in no way limiting. The scope of one or more embodiments of the claimed invention is defined not by the aforementioned descriptions but by the appended claims. While the claimed invention has been described in detail with reference to specific embodiments, it is clear to those of ordinary skill in the art that many variations and modifications can be made without departing from the essence and scope of the claimed invention.

What is claimed is:

1. A method, comprising:
measuring a blood pressure of a person;
determining an inaccuracy range of the measured blood pressure;
determining whether the person is in a cardiovascular risk category based on the measured blood pressure and the inaccuracy range; and
displaying the whether the person is in the cardiovascular risk category.

2. The method of claim 1, further comprising prompting to maintain, re-calibrate or replace a blood pressure measurement apparatus used for measuring the blood pressure of the person when the inaccuracy range exceeds a predetermined value.

3. The method of claim 1, wherein determining whether the person is in a cardiovascular risk category comprises determining whether a predetermined threshold for the cardiovascular risk category is within the inaccuracy range of the measured blood pressure.

4. The method of claim 3, wherein the cardiovascular risk category is a first cardiovascular risk category, the predetermined threshold is a first predetermined threshold, and further comprising determining whether the person is in a second cardiovascular risk category by determining whether a second predetermined threshold for the second cardiovascular risk category is within the inaccuracy range of the measured blood pressure.

5. The method of claim 1, wherein the inaccuracy range comprises a predetermined percentage of confidence.

6. The method of claim 1, wherein the inaccuracy range comprises a predetermined amount above or below the measured blood pressure.

7. The method of claim 1, wherein the inaccuracy range comprises a statistical measure.

8. An apparatus, comprising:
a blood pressure interface unit;
a measurement unit coupled to the blood pressure interface unit; and
a display/control unit in communication with the measurement unit,
wherein:
the blood pressure interface unit is to measure the blood pressure of a person,
the measurement unit is to determine an inaccuracy range of the measured blood pressure, and
the display/control unit is to display a cardiovascular risk category of the person on the basis of the measured blood pressure and inaccuracy range.

9. The apparatus of claim 8, wherein the display/control unit is further to display a prompt to maintain or re-calibrate or replace the blood pressure interface and/or measurement unit when the inaccuracy value exceeds a predetermined value.

10. The apparatus of claim 8, wherein the display/control unit comprises a mobile system such as a tablet computing device or mobile phone or smart phone, with communication to the measurement unit via direct cable or by wireless means.

11. The apparatus of claim 8, wherein power is also provided by the mobile system.

12. The apparatus of claim 8, wherein the apparatus is to determine whether the person is in the cardiovascular risk category based on whether a predetermined threshold for the cardiovascular risk category is within the inaccuracy range of the measured blood pressure.

13. The apparatus of claim 12, wherein the cardiovascular risk category is a first cardiovascular risk category, the predetermined threshold is a first predetermined threshold, and wherein the apparatus is further to determine whether the person is in a second cardiovascular risk category based on whether a second predetermined threshold for the second cardiovascular risk category is within the inaccuracy range of the measured blood pressure.

14. The apparatus of claim 8, wherein the display/control unit is to display the cardiovascular risk category using a color or pattern, and is to display the inaccuracy value using a variable box fill amount.

15. A non-transitory computer readable medium (CRM) comprising instructions executable by a processor on an apparatus that, when executed, cause the apparatus to:
measure a blood pressure of a person;
determine an inaccuracy range of the measured blood pressure;
determine whether the person is in a cardiovascular risk category based on the measured blood pressure and the inaccuracy range; and
display whether the person is in the cardiovascular risk category

16. The CRM of claim 15, wherein the instructions are further to cause the apparatus to determine whether the person is in a cardiovascular risk category based on whether a predetermined threshold for the cardiovascular risk category is within the inaccuracy range of the measured blood pressure.

17. The CRM of claim 16, wherein the cardiovascular risk category is a first cardiovascular risk category, the predetermined threshold is a first predetermined threshold, and the instructions are further to cause the apparatus to determine whether the person is in a second cardiovascular risk category based on whether a second predetermined threshold for the second cardiovascular risk category is within the inaccuracy range of the measured blood pressure.

18. The CRM of claim 15, wherein the inaccuracy range comprises a predetermined percentage of confidence.

19. The CRM of claim 15, wherein the inaccuracy range comprises a predetermined amount above or below the measured blood pressure.

20. The CRM of claim 19, wherein the predetermined amount above or below is one standard deviation.
